(19) United States
(12) Patent Application Publication
(10) Pub. No.: US 2017/0231509 A1
(43) Pub. Date: Aug. 17, 2017

(54) MEASUREMENT APPARATUS AND MEASUREMENT METHOD

(71) Applicant: KYOCERA Corporation, Kyoto (JP)

(72) Inventor: Takahiro WATANABE, Yokohama-shi, Kanagawa (JP)

(73) Assignee: KYOCERA Corporation, Kyoto (JP)

(21) Appl. No.: 15/502,011

(22) PCT Filed: Aug. 24, 2015
(86) PCT No.: PCT/JP2015/004231

§ 371 Date: Feb. 6, 2017

(30) Foreign Application Priority Data
Aug. 27, 2014 (JP) 2014-172865

(51) Int. Cl. A61B 5/026; A61B 5/00

(52) U.S. Cl. CPC A61B 5/0261; A61B 5/6898

(57) ABSTRACT

A measurement apparatus includes a contact unit for contacting with a test site, a biological sensor for acquiring a biometric output from the test site, an inclination detection unit for detecting an inclination of the measurement apparatus, and a controller. The controller generates biological information based on the biometric output from the biological sensor when the inclination detected by the inclination detection unit falls within a predetermined angular range in a state where the test site is in contact with the contact unit while supporting the measurement apparatus in an inclining manner.

### CROSS REFERENCE TO RELATED APPLICATION

This application claims priority to and the benefit of Japanese Patent Application No. 2014-172865 (filed on Aug. 27, 2014), the entire contents of which are incorporated herein by reference.

### TECHNICAL FIELD

This disclosure relates to a measurement apparatus and a measurement method.

### BACKGROUND

Conventionally, there is known a measurement apparatus for measuring biological information by acquiring a biological information output from a test site such as a subject's (user's) fingertip.

### SUMMARY

A measurement apparatus of the disclosure includes:

a contact unit for contacting with a test site;

a biological sensor for acquiring a biometric output from the test site;

an inclination detection unit for detecting an inclination of the measurement apparatus; and

a controller, wherein

the controller generates biological information based on the biometric output from the biological sensor when the inclination detected by the inclination detection unit falls within a predetermined angular range in a state where the test site is in contact with the contact unit while supporting the measurement apparatus in an inclining manner.

It should be understood that a method substantially corresponding to the above measurement apparatus may also implement the disclosure and thus is included in the scope of the disclosure.

For example, a measurement method of the disclosure includes:

a step in which an inclination detection unit detects an inclination of a measurement apparatus when a test site is in contact with a contact unit while supporting the measurement apparatus in an inclining manner; and

a step of generating biological information based on a biometric output acquired by measuring the test site with the biological sensor when the inclination detected by the inclination detection unit falls within a predetermined angular range.

### BRIEF DESCRIPTION OF THE DRAWINGS

In the accompanying drawings:

FIG. 1 is a functional block diagram illustrating a schematic configuration of a measurement apparatus according to a first embodiment;

FIG. 2 is an external perspective view illustrating a schematic configuration of the measurement apparatus of FIG. 1 when viewed from a rear side thereof;

FIG. 3 is a diagram schematically illustrating an example of a using mode of the measurement apparatus of FIG. 1;

FIG. 4 is a flowchart illustrating an example of control for measurement of biological information performed by a controller;

FIG. 5 is a functional block diagram illustrating a schematic configuration of a measurement apparatus according to a second embodiment;

FIG. 6 is an external perspective view illustrating a schematic configuration of the measurement apparatus of FIG. 5 when viewed from a rear side thereof;

FIG. 7 is a diagram illustrating an example of guide information presented by a guide unit;

FIG. 8 is a flowchart illustrating an example of control performed by the controller to guide a test site;

FIG. 9 is a diagram illustrating an example variation of a guide; and

FIG. 10 is a diagram schematically illustrating another example of the using mode of the measurement apparatus.

### DETAILED DESCRIPTION

A conventional blood flow measurement apparatus for measuring a blood flow as the biological information emits laser light to the fingertip and measures the blood flow based on scattered light from the blood flowing in the capillaries at the fingertip.

Since a result of measurement of the biological information varies depending on a pressure applied to the measurement apparatus by the test site, maintaining a constant pressure may enhance measurement accuracy of the biological information. However, it is not easy to maintain a constant pressure applied to the measurement apparatus.

Therefore, it could be helpful to provide a measurement apparatus and a measurement method which are capable of enhancing the measurement accuracy of the biological information.

Hereinafter, embodiments of the disclosure will be described in detail with reference to the drawings.

### First Embodiment

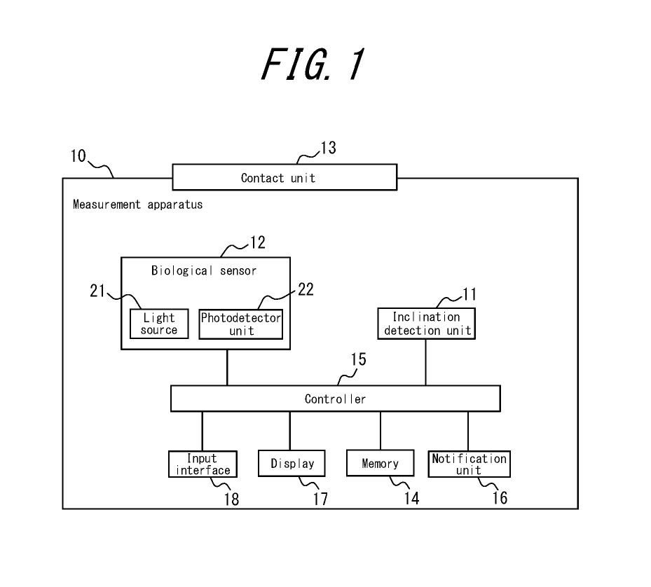

FIG. 1 is a functional block diagram illustrating a schematic configuration of a measurement apparatus according to a first embodiment. A measurement apparatus 10 includes an inclination detection unit 11, a biological sensor 12, a contact unit 13, a memory 14, a controller 15, a notification unit 16, a display 17, and an input interface 18. In the present embodiment, the measurement apparatus 10 is implemented by a smartphone (a multi-function mobile phone). The measurement apparatus 10 includes the display 17 on a front surface of the smartphone and a contact unit 13 on a rear surface opposite to the front surface.

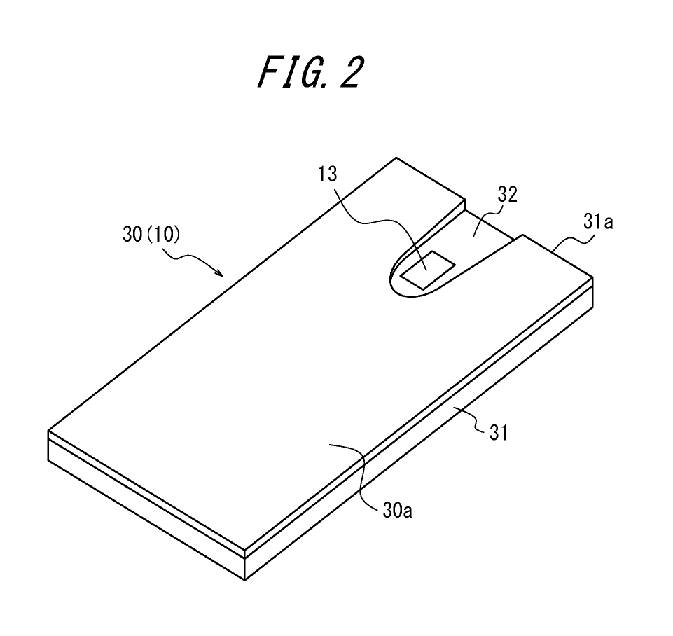

FIG. 2 is an external perspective view illustrating a schematic configuration of the measurement apparatus 10 of FIG. 1 when viewed from a rear side thereof. As illustrated in FIG. 2, a smartphone 30 includes, on a rear surface 30a thereof, a recess 32 extending from a central portion of one end (an upper end 31a) of a housing 31 toward a central portion of the housing 31. The recess 32 defines a contact position of the test site when a subject measures biological information with the measurement apparatus 10. The recess 32 is provided with the contact unit 13 for allowing the test site to contact therewith. The subject measures the biological information with the measurement apparatus 10 by contacting the finger serving as the test site with the contact unit 13. Note that a mechanism to define the contact position of the test site for measurement of the biological information using the measurement apparatus 10 by the subject is not limited to the recess 32. Such a mechanism may be, for example, a rib, one or more protrusions, and a groove.

The biological information measured by the measurement apparatus 10 may be any biological information that may be measured by the biological sensor 12. The present embodiment will be described assuming that the measurement apparatus 10 measures, by way of example, a blood flow of the subject serving as information on a flow of blood.

In FIG. 1, the inclination detection unit 11 detects an inclination of the measurement apparatus 10 with respect to, for example, a horizontal direction or a vertical direction. The inclination detection unit 11 is a known acceleration sensor of, for example, a piezo-resistive type, a capacitive type, or a thermal detection type. The inclination detection unit 11 transmits information on the inclination of the measurement apparatus 10 thus detected to the controller 15.

The biological sensor 12 acquires a biometric output from the test site. When the measurement apparatus 10 measures the blood flow as described in the present embodiment, the biological sensor 12 includes a light source 21 and a photodetector unit 22.

The light source 21 emits laser light when controlled by the controller 15. For example, the light source 21 emits, as measurement light, the laser light in a wavelength which allows detection of a predetermined component contained in the blood and may be, for example, an LD (Laser Diode).

The photodetector unit 22 receives, as the biometric output, scattered light of the measurement light from the test site. The photodetector unit 22 may be, for example, a PD (Photodiode). The biological sensor 12 transmits a photoelectric conversion signal of the scattered light received by the photodetector unit 22 to the controller 15.

The contact unit 13 is a unit with which the test site such as a subject's finger comes into contact for the measurement of the biological information. The contact unit 13 may be, for example, a plate-like member. The contact unit 13 is made of a transparent member to receive at least the measurement light from the light source 21 and the scattered light from the test site.

The memory 14 may be a memory such as a semiconductor memory and a magnetic memory and stores various information and programs for operating the measurement apparatus 10 and also functions as a work memory. The memory 14 stores, for example, a predetermined angular range used as a criterion as to whether the inclination of the measurement apparatus 10 is suitable for the measurement of the biological information.

The controller 15 is a processor for controlling and managing the measurement apparatus 10 in its entirety including each functional block of the measurement apparatus 10. The controller 15 is a processor such as CPU (Central Processing Unit) for executing a program defining a control procedure stored in, for example, the memory 14 or an external storage medium.

The controller 15 controls the notification unit 16 such that the notification unit 16 notifies of information on the inclination of the measurement apparatus 10 detected by the inclination detection unit 11. The information on the inclination includes, for example, information whether the inclination detected by the inclination detection unit 11 falls within the predetermined angular range stored in the memory 14. The information on the inclination, when the inclination detected by the inclination detection unit 11 falls out of the predetermined angular range, may include, for example, an instruction for the subject what to do to have the inclination of the measurement apparatus 10 within the predetermined angular range. The instruction for the subject includes, for example, an instruction to increase or decrease the inclination of the measurement apparatus 10.

The notification unit 16 may issue the notification, for example, in a visual manner using an image, a character, and lighting, in an auditory manner using a voice and the like, or a combination thereof. The notification unit 16, in order to notify in the visual manner, displays, for example, the image or the character in a display device such as the display 17. The notification unit 16 may notify by, for example, turning on a light emitter such as an LED. The notification unit 16, in order to notify in the auditory manner, outputs, for example, an alarm sound, a voice guidance, and the like from a sound generating device such as a speaker. The notification issued by the notification unit 16 may include notification of start of the measurement of the biological information such as a voice guide stating “Measurement will start in this position”. The notification issued by the notification unit 16 is not limited to the visual manner nor the auditory manner but may by any manner perceivable by the subject. When the measurement apparatus 10 is the smartphone 30 as described in the present embodiment, the controller 15 may use a notification function unit of the smartphone 30 as the notification unit 16.

The controller 15 controls the measurement light emitted from the light source 21. For example, when the measurement apparatus 10 is set to be ready for the measurement of the biological information by an operation of the subject in a state where the controller 15 determines that the inclination of the measurement apparatus 10 detected by the inclination detection unit 11 falls within the predetermined angular range, the controller 15 controls the light source 21 such that the light source 21 emits the laser light serving as the measurement light. The controller 15, when the measurement light is emitted, starts acquiring the biometric output from the biological sensor 12.

The controller 15, after the light source 21 emits the laser light, determines whether the biological sensor 12 has completed acquiring the biometric information. The controller 15 may determine that the acquisition of the biometric output is completed when, for example, a predetermined time period has elapsed after the biological sensor 12 starts acquiring the biometric output. Alternatively, the controller 15 may determine that the acquisition of the biometric output is completed when, for example, the biological sensor 12 acquires the biometric output in a sufficient quantity for the measurement of the biological information.

The controller 15, while the biological sensor 12 is acquiring the biometric output, may continue to acquire the information on the inclination of the measurement apparatus 10 detected by the inclination detection unit 11. When the controller 15 determines that, based on the information on the inclination continuously acquired, the inclination of the measurement apparatus 10 falls out of the predetermined angular range, the controller 15 may stop the emission of the measurement light from the light source 21. While the inclination of the measurement apparatus 10 changes within the predetermined angular range, the controller 15 may control the light source 21 such that the light source 21 continues to emit the measurement light.

When the controller 15 determines that the acquisition of the biometric output is completed, the controller 15 stops the emission of the laser light from the light source 21. The controller 15 generates the biological information based on an output (a biological information output) from the photodetector unit 22. Although in the measurement apparatus 10 of the present embodiment the controller 15 generates the biological information, a function unit independent of the controller 15 may generate the biological information.

Here, a blood flow measuring technology using a Doppler shift employed by the controller 15 will be described. The controller 15, in order to measure the blood flow, controls the light source 21 such that the light source 21 emits the laser light into tissues (the test site) of a living body such that the photodetector unit 22 receives the scattered light from the inside of the tissues of the living body. Then, the controller 15 calculates the blood flow based on an output associated with the scattered light received.

Inside the tissues of the living body, the scattered light scattered by the blood cells which are moving are subjected to a frequency shift (the Doppler shift) due to Doppler effect which increases in proportion to a moving speed of the blood cells in the blood. The controller 15 detects an Unari-signal (also referred to as a beat signal) generated by optical interference between the scattered light from still tissues and the scattered light from the blood cells which are moving. The beat signal indicates intensity represented by a time function. Then, the controller 15, from the beat signal, generates a power spectrum in which power is represented by a frequency function. In the power spectrum of the beat signal, the Doppler shift frequency increases in proportion to the moving speed of the blood cells, and the power corresponds to an amount of the blood cells. Then, the controller 15 calculates the blood flow by multiplying the power spectrum of the beat signal by the frequency and integrating.

The controller 15 may store the biological information, generated based on the biometric output acquired by the biological sensor 12, in the memory 14.

The display 17 is a display device such as a liquid crystal display, an organic EL display, and an inorganic EL display. The display 17 displays, for example, a result of the measurement of the bio-information conducted by the measurement apparatus 10. The display 17 may also function as the notification unit 16 by displaying the information on the inclination of the measurement apparatus 10.

The input interface 18 receives an input operation from the subject and is, for example, an operation button (an operation key). The input interface 18 may be a touch panel and displayed in a portion of the display 17 so as to receive a touch input operation from the subject. The subject may, for example, run a dedicated application for the measurement of the biological information by operating the input interface 18.

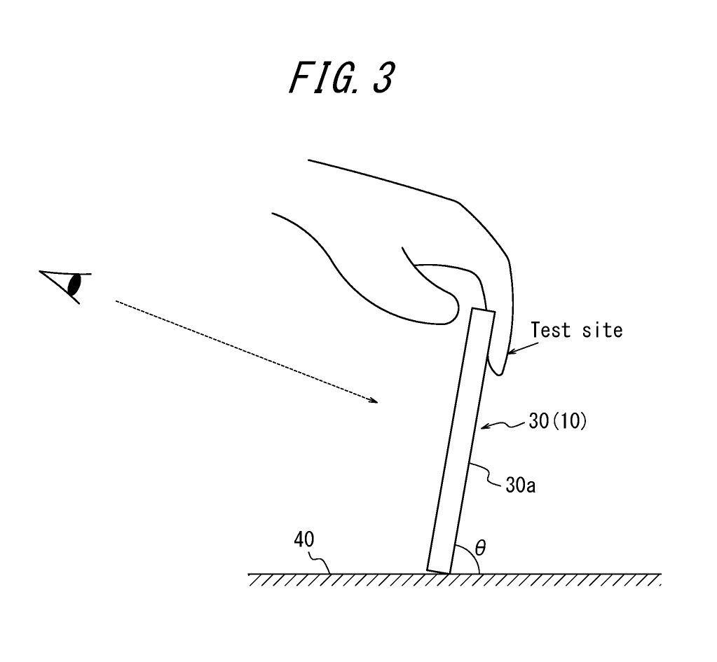

Next, a method of measuring the biological information with the measurement apparatus 10 of the present embodiment will be described. FIG. 3 is a diagram schematically illustrating an example of a using mode of the measurement apparatus 10 of FIG. 1. The subject, after operating the measurement apparatus 10 to set the measurement apparatus 10 to be ready for the measurement of the biological information, places the smartphone 30 on a placing surface 40, which is a horizontal surface of a desk or the like, in such a manner that the front surface of the smartphone 30 faces the subject as illustrated in FIG. 3. In this state, the subject may view an image displayed in the display 17 on the front surface of the smartphone 30. At this time, a lower end opposite to the upper end 31a of the housing 31 is in contact with the placing surface 40. The test site is positioned at a contact position defined by the recess 32 and in contact with the contact unit 13 on the housing 31. That is, in measuring the biological information, the smartphone 30 is supported by the placing surface 40 and the test site positioned at the contact unit 13 in such a manner as to incline with respect to the placing surface 40 at the lower end of the smartphone 30. When the smartphone 30 is supported in this manner, the inclination of the measurement apparatus 10, i.e., an angle θ between the placing surface 40 and the rear surface 30a is detected by the inclination detection unit 11. The subject may adjust the inclination of the measurement apparatus 10 by adjusting a height of the test site while keeping the test site in contact with the contact unit 13.

The controller 15 generates the biological information based on the output of the photodetector unit 22 when the angle 0 detected by the inclination detection unit 11 falls within the predetermined angular range. Here, the controller 15 generates the biological information based on the output of the photodetector unit 22 when, for example, the angle θ falls within a range between θ₁ and θ₂ (0°<θ₁<θ₂90°).

Here, both weight of the measurement apparatus 10 and a position of the contact unit 13 where the test site supports the measurement apparatus 10 are fixed. In a stationary state, therefore, a load applied to the housing 31 by the test site (i.e., a pressure applied to the contact unit 13 by the test site) and a load applied to the test site by the measurement apparatus 10 are constant. Accordingly, the load applied to the test site by the measurement apparatus 10 is uniquely determined by the angle θ. Therefore, provided that F₁ represents the load applied to the test site by the measurement apparatus 10 when the angle θ is θ₁, and F₂ represents the load applied to the test site by the measurement apparatus 10 when the angle θ is θ₂, the controller 15 generates the biological information based on the output from the photodetector unit 22 when the load applied to the test site is between F₁ and F₂.

That is, in the measurement apparatus 10, when values of the θ₁ and the θ₂ for making a range of the load determined by the F₁ and the F₂ within a range suitable for the measurement of the biological information are stored in the memory 14, the subject may measure the biological information when the load applied to the test site falls within a predetermined load range. The predetermined load range may be, for example, a load range in which the load applied to the test site is suitable for the measurement of the blood flow. The load range suitable for the measurement of the blood flow is, for example, a load range in which an error of the result of the measurement of the blood flow falls within a predetermined error range based on a statistical relationship between the load and a measurement error. This measurement method using the measurement apparatus 10 enables, regardless of differences between subjects, the measurement of the biological information when the load applied to the test site falls within the predetermined load range. Therefore, measurement accuracy of the biological information may be enhanced.

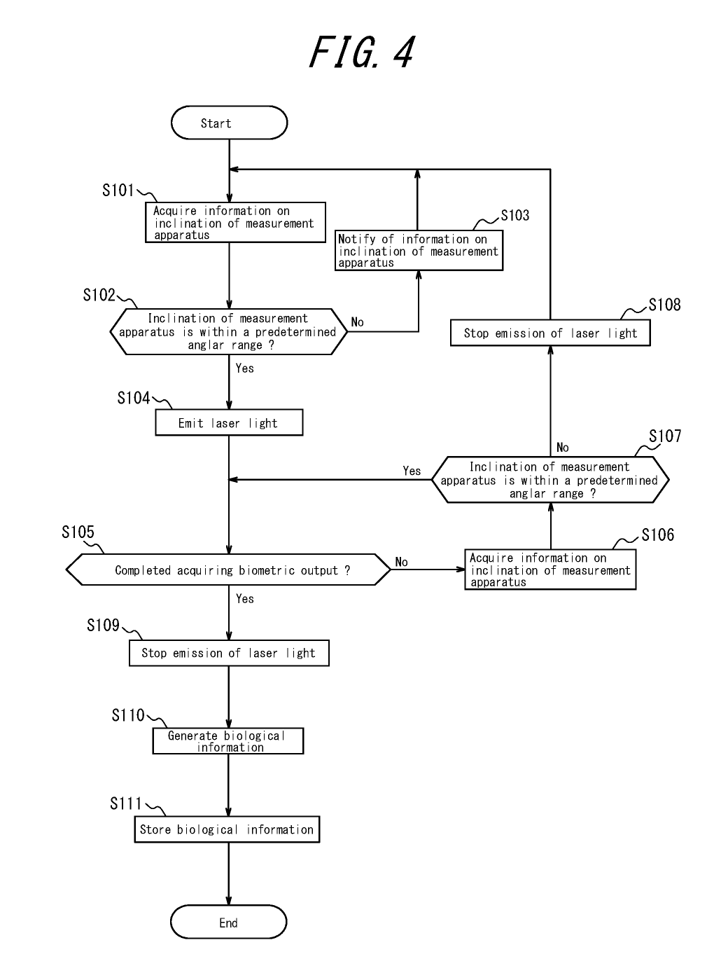

Next, an example of control for the measurement of the biological information performed by the controller 15 will be described with reference to a flowchart illustrated in FIG. 4. A flow illustrated in FIG. 4 starts when, for example, the measurement apparatus 10 is set to be ready for the measurement of the blood flow by an operation to the measurement apparatus 10. The subject, after setting the measurement apparatus 10 to be ready for the measurement of the blood flow, supports the measurement apparatus 10 while maintaining the test site in contact with the contact unit 13 as described above with reference to FIG. 3. At the start of this flow, the light source 21 does not emit the laser light.

The controller 15 acquires the information on the inclination of the measurement apparatus 10 detected by the inclination detection unit 11 (step S101).

The controller 15, based on the information on the inclination, determines whether the inclination of the measurement apparatus 10 falls within the predetermined angular range (step S102).

When the controller 15 determines that the inclination of the measurement apparatus 10 falls out of the predetermined angular range (No at step S102), the controller 15 controls the notification unit 16 such that the notification unit 16 notifies of the information on the inclination of the measurement apparatus 10 (step S103). When the subject notices the notification, the subject may adjust the inclination of the measurement apparatus 10. The controller 15 then returns to step S101 and acquires the information on the inclination of the measurement apparatus 10 adjusted by the subject.

When the controller 15 determines that the inclination of the measurement apparatus 10 falls within the predetermined angular range (Yes at step S102), the controller 15 controls the light source 21 such that the light source 21 emits the laser light (step S104). When the laser light is emitted, the biological sensor 12 starts acquiring the biometric output.

Next, the controller 15 determines whether the biological sensor 12 has completed in acquiring the biometric output (step S105).

When the controller 15 determines that the acquisition of the biometric output is not completed (No at step S105), acquires the information on the inclination of the measurement apparatus 10 from the inclination detection unit 11 (step S106).

The controller 15, based on the information on the inclination, determines whether the inclination of the measurement apparatus 10 is maintained within the predetermined angular range (step S107).

When the controller 15 determines that the inclination of the measurement apparatus 10 is maintained within the predetermined angular range (Yes at step S107), the controller 15 returns to step S105.

On the other hand, when the controller 15 determines that the inclination of the measurement apparatus 10 is not maintained within the predetermined angular range (No at step S107), the controller 15 stops the emission of the laser light from the light source 21 (step S108). Then, the controller 15 returns to step S101.

When the controller 15 determines at step S105 that the acquisition of the biometric output is completed (Yes at step S105), the controller 15 stops the emission of the laser light from the light source 21 (step S109). Thereby, the biological sensor 12 ends acquiring the biometric output.

Next, the controller 15 generates the biological information based on the biometric output acquired (step S110).

The controller 15 stores the biological information thus generated in the memory 14 (step S111). The controller 15 may display the biological information thus generated in the display 17 to present a result of the measurement to the subject.

In the measurement apparatus 10 of the present embodiment, as described above, the controller 15 generates the biological information based on the output of the photodetector unit 22 when the inclination of the measurement apparatus 10 detected by the inclination detection unit 11 falls within the predetermined angular range. Since the weight of the measurement apparatus 10 and the position of the contact unit 13 of the measurement apparatus 10 to contact with the test site are fixed, the inclination of the measurement apparatus 10 in measuring the biological information uniquely determines the load applied to the test site, regardless of differences between subjects. Accordingly, the measurement apparatus 10 may generate the biological information when the load applied to the test site falls within the predetermined range. Hence, the measurement accuracy of the biological information may be enhanced. Especially, the narrower the predetermined angular range stored in the memory 14, the narrower the range of the load used as a condition for the measurement of the biological information. Therefore, reproducibility of the result of the measurement when the measurement of the biological information is repetitively conducted becomes improved, facilitating obtainment of highly reliable data.

In the measurement apparatus 10, the notification unit 16 notifies of the information on the inclination of the measurement apparatus 10, thereby allowing the subject to recognize the inclination of the measurement apparatus 10 and facilitating the adjustment of the inclination such that the inclination falls within the predetermined angular range.

Although with the measurement method using the measurement apparatus 10 the subject may have a difficulty in viewing the contact position of the test site on the rear surface 30a of the measurement apparatus 10, the recess 32 aids the test site in contacting with the contact unit 13 with precision.

When the controller 15 controls the light source 21 such that the light source 21 emits the laser light when the inclination detected by the inclination detection unit 11 falls within the predetermined angular range, unnecessary power consumption may be suppressed.

### Second Embodiment

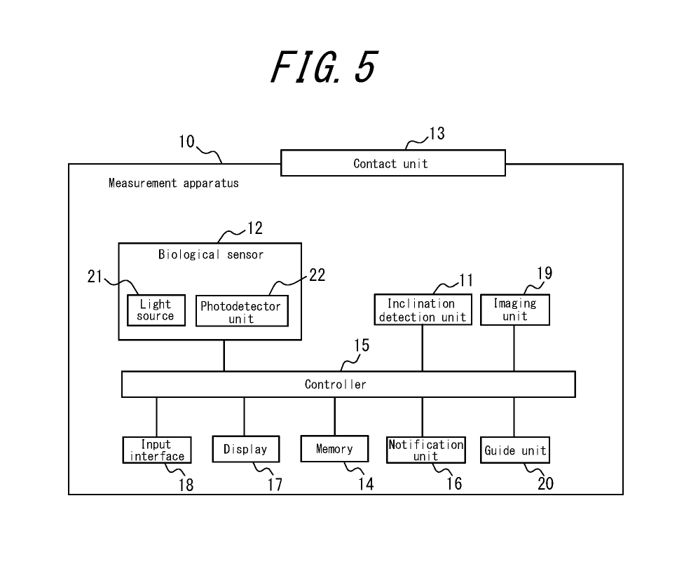

Next, a second embodiment will be described. FIG. 5 is a functional block diagram illustrating a schematic configuration of the measurement apparatus 10 according to the second embodiment. The measurement apparatus 10 of the second embodiment further includes an imaging unit 19 and a guide unit 20 in addition to the function units of the measurement apparatus 10 of the first embodiment. Hereinafter, features of the second embodiment the same as those of the first embodiment will be omitted, and different features will be described.

The imaging unit 19 captures an image on a side of the rear surface 30a of the measurement apparatus 10. The image captured by the imaging unit 19 is used by the controller 15 to determine whether the test site is in contact with the contact unit 13. Therefore, the imaging unit 19 may be arranged in the vicinity of the contact unit 13 to be able to capture an image of the test site. The imaging unit 19 may be, for example, a digital video camera.

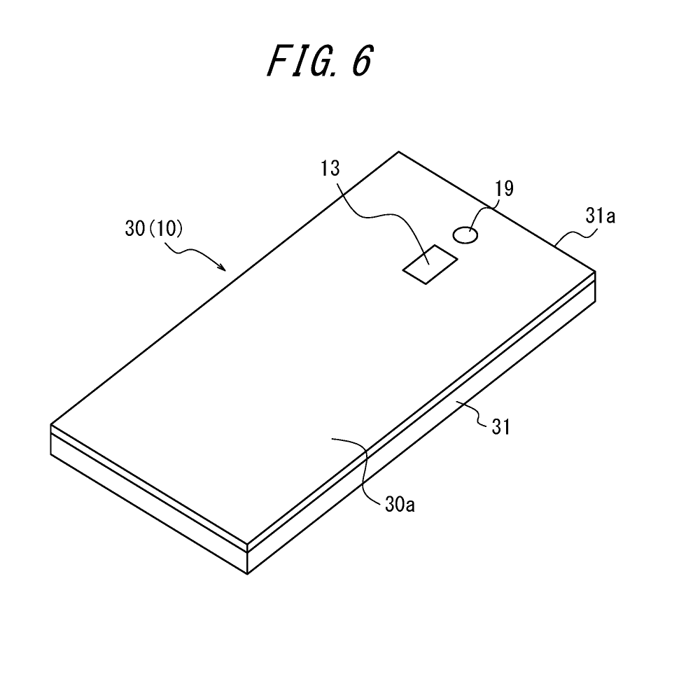

FIG. 6 is an external perspective view illustrating a schematic configuration of the measurement apparatus 10 of FIG. 5 when viewed from a rear side thereof. Unlike the measurement apparatus 10 of the first embodiment, the measurement apparatus 10 of the second embodiment does not include the recess 32 for defining the contact position of the test site. Instead, on the rear surface 30a of the measurement apparatus 10 of the second embodiment, from the central portion of the upper end 31a of the housing 31 toward the central portion of the housing 31, the imaging unit 19 and the contact unit 13 are arranged in the stated order. The arrangement of the imaging unit 19 and the contact unit 13 is illustrated by way of example, and the imaging unit 19 may be arranged at any position as long as being capable of capturing an image which enables the controller 15 to determine whether the test site is in contact with the contact unit 13. Also, the measurement apparatus 10 may include a plurality of imaging units 19.

In FIG. 5, the guide unit 20, when controlled by the controller 15, presents information (guide information) to guide the test site to the contact unit 13. The guide information, when the test site is not appropriately in contact with the contact unit 13, may include, for example, a direction in which the test site needs to move to appropriately contact with the contact unit 13. The guide information, when the test site is appropriately in contact with the contact unit 13, may include, for example, information indicating that the test site is appropriately in contact with the contact unit 13. Note that a state in which the test site is appropriately in contact with the contact unit 13 refers to a state in which the test site is in contact with the contact unit 13 in such a manner as to allowing the measurement apparatus 10 to measure the biological information of the test site at the contact unit 13. The guide unit 20, similarly to the notification unit 16 described above, may present information in, for example, the visual manner using the image, the character, the lighting, and the like, or the auditory manner using the voice and the like, or a combination thereof. The guide unit 20, in order to present the information in the visual manner, may display, for example, the image or the character in the display device such as the display 17.

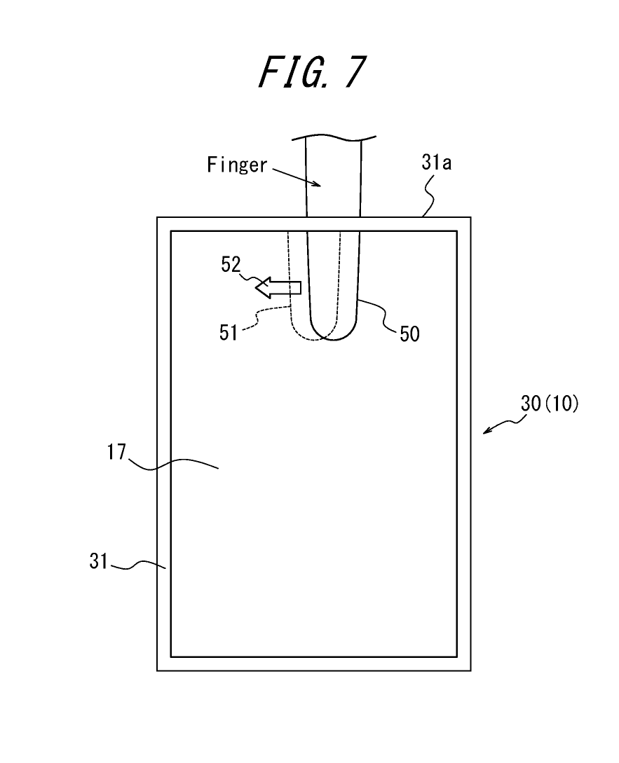

FIG. 7 is a diagram illustrating an example of the guide information presented by the guide unit 20, i.e., a schematic diagram of the smartphone 30 when viewed from a front side thereof. In FIG. 7, the display 17 used as the guide unit 20 displays the guide information. The display 17 displays a finger image 50, which is a schematic image indicative of a position of the finger in contact with the rear surface 30a of the smartphone 30. The finger image 50 is depicted in the display 17 by the controller 15 based on, for example, the image captured by the imaging unit 19. As illustrated in FIG. 7, the subject may view the subject's finger in contact with the rear surface 30a of the smartphone 30 on an extension line of the finger image 50 displayed in the display 17.

The display 17 displays, as the guide information, a target position 51 indicative of a position where the finger should contact with the rear surface 30a of the smartphone 30. The target position 51 may be indicated by, for example, a dotted line as illustrated in FIG. 7, or may be in color different from color of the finger image 50. In FIG. 7, an arrow 52 indicative of a direction in which the finger needs to move is further displayed as the guide information. The subject may adjust the position of the finger based on the finger image 50, the target position 51, and the arrow 52. For example, when the finger image 50 and the target position 51 overlap with each other, it means that the test site of the subject is appropriately in contact with the contact unit 13.

Note that the guide information is not limited to the target position 51 and the arrow 52 but may be any information for guiding the test site to the contact unit 13. Also, the number of guide information displayed in the display 17 is not limited to two, but one or more than two guide information may be displayed. Further, the guide information presented by the guide unit 20 is not limited to the visual manner nor the auditory manner but may be any manner perceivable by the subject.

In the present embodiment, the controller 15, based on the image captured by the imaging unit 19, controls the guide unit 20 such that the guide unit 20 presents the information for guiding the test site to the contact unit 13. For example, the controller 15 presumes, based on the image captured by the imaging unit 19, a position where the finger is in contact with the rear surface 30a of the smartphone 30 and controls the guide unit 20 such that the guide information is presented. For example, when the image captured by the imaging unit 19 includes a region with high illuminance (brightness), the controller 15 may consider that the contact position of the test site is deviated from the contact unit 13. The controller 15 may presume a direction of the deviation of the test site from the contact unit 13 based on, for example, a positional relationship between the contact unit 13 and the imaging unit 19 as illustrated in FIG. 6 and a position of the region with the high illuminance in the image. For example, when the illuminance of the image captured by the imaging unit 19 is lower than a predetermined value, the controller 15 may consider that the test site is appropriately in contact with the contact unit 13. When determining that the test site is in contact with the contact unit 13, the controller 15 starts the flow illustrated in FIG. 4 for the measurement of the biological information.

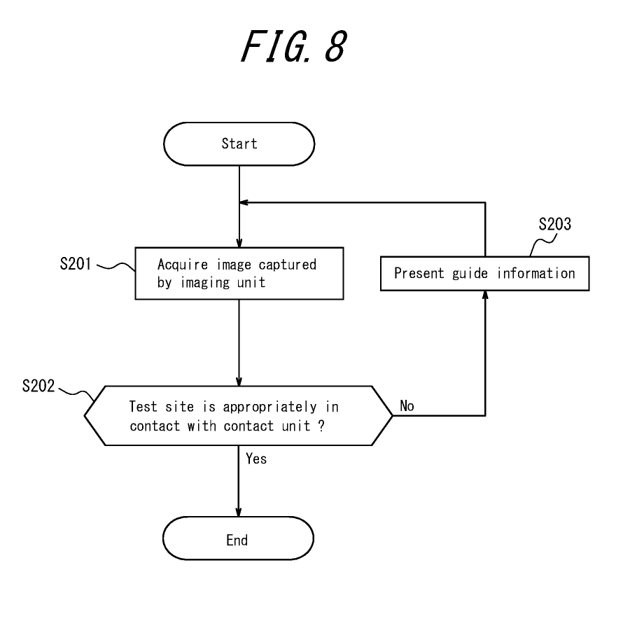

FIG. 8 is a flowchart illustrating an example of the control performed by the controller 15 to guide the test site to the contact unit 13. The flow illustrated in FIG. 8 starts when, for example, the measurement apparatus 10 is set to be ready for the measurement of the blood flow by the operation to the measurement apparatus 10. After setting the measurement apparatus 10 to be ready for the measurement of the blood flow, the subject supports the measurement apparatus 10 while maintaining the test site in contact with the contact unit 13.

First, the controller 15 acquires the image captured by the imaging unit 19 (step S201).

The controller 15, based on the image acquired, determines whether the test site is appropriately in contact with the contact unit 13 (step S202).

When the controller 15 determines that the test site is not appropriately in contact with the contact unit 13 (No at step S202), the controller 15 controls the guide unit 20 such that the guide unit 20 presents the guide information (step S203). The subject may shift the contact position of the test site in contact with the measurement apparatus 10 based on the guide information. The controller 15 returns to step 5201 and acquires the image captured by the imaging unit 19 after the subject moves the contact position of the test site.

When the controller 15 determines that the test site is appropriately in contact with the contact unit 13 (Yes at step S202), the controller 15 ends this flow. Then, the controller 15 starts the flow illustrated in FIG. 4.

In the measurement apparatus 10 of the present embodiment, the controller 15 may presume the position of the test site based on the image captured by the imaging unit 19. Therefore, when the housing 31 is not provided with the recess 32 unlike the first embodiment, the subject may appropriately bring the test site into contact with the contact unit 13.

The above embodiments should not be construed to limit the disclosure but may be modified or changed in a variety of manners. For example, functions and the like included in each constituent and step may be rearranged without logical inconsistency, so as to combine a plurality of constituents or steps together or to separate them.

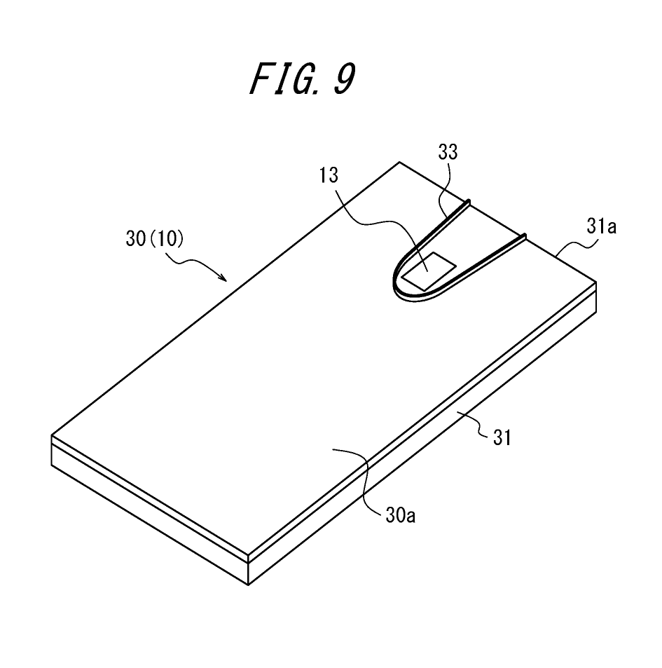

Although in the first embodiment the smartphone 30 includes the recess 32 in a portion of the housing 31, a means for defining the contact position of the test site is not limited to the recess 32. The smartphone 30 may include any guide having a function to facilitate the contact of the test site with the contact unit 13. For example, as illustrated in FIG. 9, the smartphone 30 may include, on the rear surface 30a, a guide 33 which surrounds the contact unit 13 and extends from the central portion of the upper end 31a toward the central portion of the housing 31. The subject may have the test site appropriately contact with the contact unit 13 by bringing the finger into contact with the rear surface 30a of the smartphone 30 following the guide 33.

Although in the above embodiments the measurement apparatus 10 is implemented by the smartphone 30, the measurement apparatus 10 may be implemented by other devices. For example, the measurement apparatus 10 may be implemented by, other than the smartphone 30, various devices such as a portable music player, a laptop computer, a watch, a tablet computer, and a gaming device. Also, the measurement apparatus 10 may be implemented as a dedicated device for measuring the biological information. For each device, the predetermined angular range serving as the criterion used for the measurement of the biological information by the controller 15 is appropriately determined based on factors such as the weight of each device and a position of the contact unit 13 on the device. Generally, when a center of gravity locates in a central portion of the device, the lighter the device, the smaller the predetermined angle is determined. Also, the closer to the upper end of the device, rather than the central portion of the device, the contact unit 13 is arranged, the smaller the predetermined angle is determined.

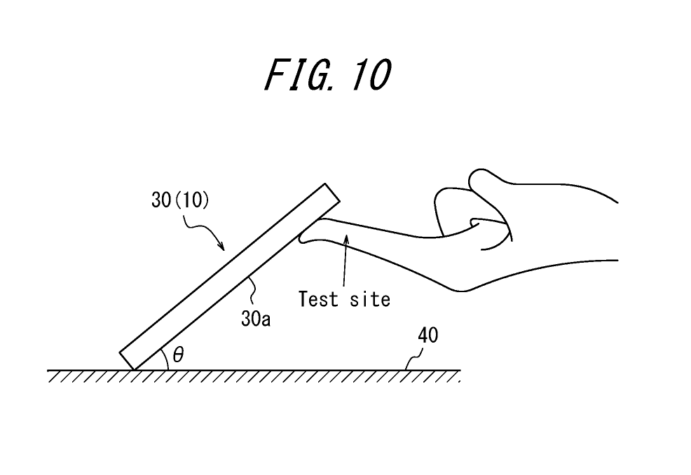

The using mode of the measurement apparatus 10 is not limited to one illustrated in FIG. 3. FIG. 10 is a diagram schematically illustrating another example of the using mode of the measurement apparatus 10. In measurement of the biological information employing the method illustrated in FIG. 10, unlike the method illustrated in FIG. 3, the subject places the smartphone 30 on the placing surface 40 with the rear surface 30a facing the subject. The subject supports the smartphone 30 with the test site in contact with the contact unit 13 in such a manner that the palm faces up. The measurement apparatus 10 measures the biological information when the inclination falls within the predetermined angular range. The measurement method illustrated in FIG. 10, as compared with the measurement method illustrated in FIG. 3, makes it easier for the subject to support the measurement apparatus 10 at a smaller inclination (a smaller angle θ). Therefore, when the measurement apparatus 10 is lightweight and therefore has a narrow predetermined angular range set thereto in order to apply a sufficient load to the test site for the measurement of the biological information, the measurement method illustrated in FIG. 10 may be effective. Note that the narrow predetermined angular range is, for example, 45° or smaller.

Although in the above embodiments the controller 15 of the measurement apparatus 10 generates the biological information based on the output of the photodetector unit 22, the generation of the biological information does not need to be performed by the controller 15 of the measurement apparatus 10. For example, a server apparatus connected to the measurement apparatus 10 via a network configured with a wired connection, a wireless connection, or a combination thereof may include a function unit having a function corresponding to the function of the controller 15 to generate the biological information and may generate the biological information. In this case, the measurement apparatus 10 acquires the information on the inclination of the measurement apparatus 10 from the inclination detection unit 11 and transmits, via a communication unit separately provided, the information on the inclination to the server apparatus. Also, the measurement apparatus 10 acquires the biological information output from the biological sensor 12 and transmits, via the communication unit separately provided, the biological information output to the server apparatus. The server apparatus generates the biological information based on the biological information output when the inclination of the measurement apparatus 10 falls within the predetermined angular range, and transmits the biological information thus generated to the measurement apparatus 10. The subject may control the measurement apparatus 10 such that the biological information received by the measurement apparatus 10 is displayed in the display 17 and thereby view the biological information. When the server apparatus generates the biological information as described above, downsizing of the measurement apparatus 10 may be achieved, as compared to the measurement apparatus 10 including all function units illustrated in FIG. 1.

1. A measurement apparatus comprising:
a contact unit for contacting with a test site;
a biological sensor for acquiring a biometric output from the test site;
an inclination detection unit for detecting an inclination of the measurement apparatus; and
a controller, wherein
the controller generates biological information based on the biometric output from the biological sensor when the inclination detected by the inclination detection unit falls within a predetermined angular range in a state where the test site is in contact with the contact unit while supporting the measurement apparatus in an inclining manner.

2. The measurement apparatus according to claim 1, wherein the biological sensor includes a light source for emitting measurement light and a photodetector for receiving scattered light of the measurement light from the test site, wherein
the controller, when the inclination detected by the inclination detection unit falls within the predetermined angular range, controls the light source such that the light source emits the measurement light, and generates the biological information based on the biometric output acquired from the photodetector unit.

3. The measurement apparatus according to claim 1, further comprising a notification unit, wherein
the controller controls the notification unit such that the notification unit notifies of information on the inclination of the measurement apparatus detected by the inclination detection unit.

4. The measurement apparatus according to claim 1, further comprising a guide for facilitating the contact of the test site with the contact unit.

5. The measurement apparatus according to claim 4, wherein the guide is a recess for defining a position to contact with the test site.

6. The measurement apparatus according to claim 1, further comprising an imaging unit for capturing an image of the test site and a guide unit, wherein
the controller, based on the image captured by the imaging unit, controls the guide unit such that the guide unit presents information for guiding the test site to the contact unit.

7. The measurement apparatus according to claim 1, wherein the biological information includes information on a blood flow.

8. A measurement method comprising:
a step in which an inclination detection unit detects an inclination of a measurement apparatus when a test site is in contact with a contact unit while supporting the measurement apparatus in an inclining manner; and
a step of generating biological information based on a biometric output acquired by measuring the test site with the biological sensor when the inclination detected by the inclination detection unit falls within a predetermined angular range.
